(19) United States
(12) Patent Application Publication
(10) Pub. No.: US 2021/0142477 A1
(43) Pub. Date: May 13, 2021

(54) Bone Age Assessment And Height Prediction Model, System Thereof And Prediction Method Thereof

(71) Applicant: China Medical University Hospital, Taichung City (TW)

(72) Inventors: Fuu-Jen Tsai, Taichung City (TW); Tzung-Chi Huang, Taichung City (TW); Ken Ying-Kai Liao, Taichung City (TW); Jiaxin Yu, Taichung City (TW)

(73) Assignee: China Medical University Hospital, Taichung City (TW)

(21) Appl. No.: 17/259,685

(22) PCT Filed: Aug. 1, 2018
(86) PCT No.: PCT/CN2018/097915

§ 371 Date: Jan. 12, 2021

(51) Int. Cl. G06T 7/00; G06K 9/42; G16H 30/20; G16H 50/20; G16H 30/40; A61B 6/00

(52) U.S. Cl. CPC G06T 7/0014; G06K 9/42; G16H 30/20; G16H 50/20; G16H 30/40; G06T 2207/10024; A61B 6/5217; G06T 2207/10116; G06T 2207/20081; G06T 2207/20084; G06T 2207/30008; A61B 6/505

(57) ABSTRACT

The present disclosure provides a bone age assessment and height prediction system including an image capturing unit and a non-transitory machine readable medium. The image capturing unit is for obtaining a target x-ray image data of a subject. The non-transitory machine-readable medium is for storing a program for assessing the development of the bones of a hand and the bone age of the subject, and predicting the adult height of the subject when executed by a processing unit. Therefore, the bone age assessment and height prediction system of the present disclosure can effectively improve the accuracy and sensitivity of the bone age assessment and the height prediction, and the time for assessing the bone age and predicting the height can be further shorten.

### RELATED APPLICATIONS

This application is a continuation of International application No. PCT/CN2018/097915, filed Aug. 1, 2018, the content of which are incorporated herein by reference.

### BACKGROUND

### Technical Field

The present disclosure relates to a medical information analysis model, system and method. More particularly, the present disclosure relates to a bone age assessment and height prediction model, a bone age assessment and height prediction system and a method for assessing a bone age and predicting a height.

### Description of Related Art

Bone age is one of the important indicators of human physiological age, and the physiological age of a person can be assessed by the process of growth, development, maturity and aging of bones. Bone age assessment (BAA) is a routine examination commonly used by pediatricians to interpret the growth and the development of children. By analyzing the different types of bones on different growth stages and by referring to the continuity and staged developmental status of human bones, the levels of growth and maturity of a subject can be assessed accurately, and the growth potential and the trend of sexual maturity of children can be further assessed and predicted accordingly.

The conventional bone age assessing method is processed by capturing the X-ray images of phalanx bones, metacarpal bones and wrist bones of a left hand or a right hand of a subject by a low-dose X-ray photographing method, and then the aforementioned X-ray images are used to assess the bone age of the subject by an atlas comparison method using Greulich and Pyle (G-P) method and Tanner-Whitehouse (TW) method. G-P method is processed by comparing the original hand bone X-ray image of the subject manually with the hand bone X-ray images of the database one-to-one according to different age ranges, and TW method is processed by selecting twenty regions of interests (ROI) of a palm and a wrist of a left-hand bone X-ray image and then comparing and analyzing one by one, and the development of the hand bone is divided into nine maturity stages so as to process the following assessment. However, when the bone assessment is processed by G-P method, the assessing result of bone age of the same subject is likely to vary due to the subjective comparison habits of different analyzers. Furthermore, although the assessing result obtaining by the TW method is more objective, the numbers of bones which should be scored are more, so that the process thereof is more complicated and more time consuming. Thus, it is unable to analyze the original bone X-ray image of the subject in a short time and then obtain a corresponding assessing result of bone age immediately.

Therefore, how to develop an assessing system of bone age and height having a high accuracy and a rapid examining speed is a technical issue with commercial values.

### SUMMARY

According to one aspect of the present disclosure is to provide a bone age assessment and height prediction model, and the bone age assessment and height prediction model is established by following steps: a reference database is obtained, an image preprocessing step is performed, a feature extracting step is performed and a training step is performed. The reference database includes a bone age-height plotted chart database and a plurality of reference hand bone X-ray image data, and each of the reference hand bone X-ray image data includes a biological age data and a gender data. An image size and a monochrome contrast of each of the reference hand bone X-ray image data are adjusted by an image data editing module in the image preprocessing step so as to obtain a plurality of normalized hand bone X-ray image data. The normalized hand bone X-ray image data are analyzed by a feature extracting module in the feature extracting step so as to obtain a plurality of image feature values, and each of the normalized hand bone X-ray image data corresponds to one of the image feature values. The image feature values are trained in the training step to achieve a convergence of the image feature values by a convolutional neural network classifier so as to obtain the bone age assessment and height prediction model, wherein the bone age assessment and height prediction model is for assessing a development of a hand bone and a bone age of a subject and predicting an adult height of the subject.

According to another aspect of the present disclosure is to provide a method for assessing a bone age and predicting a height including following steps. The bone age assessment and height prediction model according to the aforementioned aspect is provided. A target hand bone X-ray image data of the subject is provided, wherein the target hand bone X-ray image data includes a biological age data and a gender data. The target hand bone X-ray image data is preprocessed, wherein an image size and a monochrome contrast of the target hand bone X-ray image data are adjusted by the image data editing module so as to obtain a normalized target hand bone X-ray image data. The normalized target hand bone X-ray image data is analyzed by the feature extracting module so as to obtain at least one image feature value. The image feature value is analyzed by the bone age assessment and height prediction model so as to assess the development of the hand bone and the bone age of the subject and predicting the adult height of the subject.

According to further another aspect of the present disclosure is to provide a bone age assessment and height prediction system including an image capturing unit and a non-transitory machine readable medium. The image capturing unit is for obtaining a target hand bone X-ray image data of a subject, wherein the target hand bone X-ray image data includes a biological age data and a gender data. The non-transitory machine readable medium is signally connected to the image capturing unit, wherein the non-transitory machine readable medium is for storing a program, when the program executed by a processor is for assessing a development of a hand bone and a bone age of a subject and predicting an adult height of the subject, and the program includes a reference database obtaining module, a first image data editing module, a feature extracting module, a training module, a second image data editing module, a target feature extracting module and a comparing module. The reference database obtaining module is for obtaining a reference database, and the reference database includes a bone age-height plotted chart database and a plurality of reference hand bone X-ray image data, wherein each of the reference hand bone X-ray image data includes a biological age data and a gender data. The first image data editing module is for adjusting an image size and a monochrome contrast of each of the reference hand bone X-ray image data so as to obtain a plurality of normalized hand bone X-ray image data. The feature extracting module is for analyzing the normalized hand bone X-ray image data so as to obtain a plurality of reference image feature values, and each of the normalized hand bone X-ray image data corresponds to one of the reference image feature values. The training module is for training the reference image feature values to achieve a convergence of the reference image feature values by a convolutional neural network classifier so as to obtain a bone age assessment and height prediction model. The second image data editing module is for adjusting an image size and a monochrome contrast of the target hand bone X-ray image data so as to obtain a normalized target hand bone X-ray image data. The target feature extracting module is for analyzing the normalized target hand bone X-ray image data so as to obtain at least one target image feature value. The comparing module is for analyzing the at least one target image feature value by the bone age assessment and height prediction model so as to obtain a target image feature weight data, and the target image feature weight data is compared to the bone age-height plotted chart database of the reference database so as to output an assessing result of the development of the hand bone of the subject, an assessing result of the bone age of the subject and a predicting result of an adult height of the subject.

### BRIEF DESCRIPTION OF THE DRAWINGS

The present disclosure can be more fully understood by reading the following detailed description of the embodiment, with reference made to the accompanying drawings as follows:

FIG. 1 is an establishing flow chart of a bone age assessment and height prediction model according to one embodiment of the present disclosure.

FIG. 2 is a flow chart of a method for assessing a bone age and predicting a height according to another embodiment of the present disclosure.

FIG. 3 is a block diagram of a bone age assessment and height prediction system according to further another embodiment of the present disclosure.

FIG. 4 is a partial establishing flow chart of a bone age assessment and height prediction model of the present disclosure.

FIG. 5 is a block diagram of a convolutional neural network classifier of the bone age assessment and height prediction model of the present disclosure.

FIG. 6 shows an applying result of the bone age assessment and height prediction system of the present disclosure.

### DETAILED DESCRIPTION

The present disclosure will be further exemplified by the following specific embodiments. However, the readers should understand that the present disclosure should not be limited to these practical details thereof, that is, in some embodiments, and these practical details are used to describe how to implement the materials and methods of the present disclosure and are not necessary.

Please refer to FIG. 1, which is an establishing flow chart of a bone age assessment and height prediction model 100 according to one embodiment of the present disclosure. The bone age assessment and height prediction model 100 is for assessing a development of a hand bone and a bone age of a subject and predicting an adult height of the subject, and the steps for establishing the bone age assessment and height prediction model 100 include Step 110, Step 120, Step 130 and Step 140.

In Step 110, a reference database is obtained, wherein the reference database includes a bone age-height plotted chart database and a plurality of reference hand bone X-ray image data, wherein each of the reference hand bone X-ray image data includes a biological age data and a gender data. More preferably, each of the reference hand bone X-ray image data can be a reference hand bone X-ray image data of a non-dominant hand so as to prevent the accuracy of the assessing results of the bone age assessment and height prediction model 100 of the present disclosure from affecting by the bone morphological variation caused by the using frequency and the using habits of the dominant hand.

More preferably, a format of each of the reference hand bone X-ray image data can be a DICOM (DICOM) format so as to store the biological age data, the gender data and other basic information of each of the reference hand bone X-ray image data in the header files (header) of each of the reference hand bone X-ray image data and then facilitate the following analysis. Furthermore, because the physiological maturity of males and females are not the same, the developmental morphology of bones and their corresponding physiological ages are also different, so that the bone age assessment and height prediction model 100 of the present disclosure can analyze the reference hand bone X-ray image data of different genders by processing the feature extracting step and the training step, respectively so as to assess and predict the gendered development of the hand bone, the bone age and the adult height of the subject. More preferably, the bone age-height plotted chart database can include a male bone age-height plotted chart sub-database and a female bone age-height plotted chart sub-database so as to analyze the subjects of different genders.

In Step 120, an image preprocessing step is performed, wherein an image size and a monochrome contrast of each of the reference hand bone X-ray image data are adjusted by an image data editing module so as to obtain a plurality of normalized hand bone X-ray image data. In detail, the image data editing module can respectively adjust the image size of different reference hand bone X-ray image data into 256 pixels×256 pixels and adjust the monochrome contrast thereof, so that the color difference of black and white of different reference hand bone X-ray image data can be reduced and the image clarity can be enhanced so as to facilitate the following analysis.

Furthermore, in Step 120, each of the reference hand bone X-ray image data can be further processed by a color gamut expansion process. In detail, the image data editing module can calculate a gray level of each of the reference hand bone X-ray image data and automatically fill colors into pixel rows and pixel columns of image of each of the reference hand bone X-ray image data according to the aforementioned calculating results so as to transform each of the reference hand bone X-ray image data being monochrome into multicolor. Thus, it is favorable for enhancing the accuracy of the following analysis, but the present disclosure is not limited to the aforementioned description and the disclosure content of the drawings.

In Step 130, a feature extracting step is performed, wherein the normalized hand bone X-ray image data are analyzed by a feature extracting module so as to obtain a plurality of image feature values, and each of the normalized hand bone X-ray image data corresponds to one of the image feature values. In detail, the bone age assessment and height prediction model 100 of the present disclosure can automatically analyze the image information of the normalized hand bone X-ray image data and obtain the corresponding image feature value by the feature extracting module, so that the assessing and predicting efficiency of the bone age assessment and height prediction model 100 of the present disclosure can be enhanced.

In Step 140, a training step is performed, wherein the image feature values are trained to achieve a convergence of the image feature values by a convolutional neural network classifier so as to obtain the bone age assessment and height prediction model 100. More preferably, the convolutional neural network classifier can be Inception-ResNet-v2 convolutional neural network classifier. The Inception-ResNet-v2 convolutional neural network classifier is a large scale visual recognition convolutional neural network classifier based on ImageNet visual image database, and the training depth of the convolutional neural network thereof can be effectively expanded by the arrangement of the residual connections. Thus, Inception-ResNet-v2 convolutional neural network classifier has a quite high accuracy applied in the classification and the recognition of images.

Please refer to FIG. 2, which is a flow chart of a method 200 for assessing a bone age and predicting a height according to another embodiment of the present disclosure. The method 200 for assessing the bone age and predicting the height includes Step 210, Step 220, Step 230, Step 240 and Step 250.

In Step 210, a bone age assessment and height prediction model is provided, and the bone age assessment and height prediction model is established by the aforementioned Step 110 to Step 140.

In Step 220, a target hand bone X-ray image data of a subject is provided, wherein the target hand bone X-ray image data includes a biological age data and a gender data. More preferably, the target hand bone X-ray image data can be a target hand bone X-ray image data of a non-dominant hand so as to prevent the accuracy of the assessing results of the method 200 for assessing the bone age and predicting the height of the present disclosure from affecting by the bone morphological variation caused by the using frequency and the using habits of the dominant hand.

More preferably, a format of the target hand bone X-ray image data can be a DICOM format so as to store the biological age data, the gender data and other basic information of the target hand bone X-ray image data in the header files of the target hand bone X-ray image data and then facilitate the following analysis. Furthermore, because the physiological maturity of males and females are not the same, the developmental morphology of bones and their corresponding physiological ages are also different, so that the method 200 for assessing the bone age and predicting the height of the present disclosure can analyze the target hand bone X-ray image data of different genders so as to assess and predict the gendered development of the hand bone, the bone age and the adult height of the subject.

In Step 230, the target hand bone X-ray image data is preprocessed, wherein an image size and a monochrome contrast of the target hand bone X-ray image data are adjusted by the image data editing module so as to obtain a normalized target hand bone X-ray image data. In detail, the image data editing module can adjust the image size of the target hand bone X-ray image data into 256 pixels×256 pixels and adjust the monochrome contrast thereof, so that the monochrome contrast thereof can be adjusted and the image clarity can be enhanced so as to facilitate the following analysis.

Furthermore, in Step 230, the target hand bone X-ray image data can be further processed by a color gamut expansion process by the image data editing module. In detail, the image data editing module can calculate the gray level of the target hand bone X-ray image data and automatically fill colors into pixel rows and pixel columns of image of the target hand bone X-ray image data according to the aforementioned calculating results so as to transform the target hand bone X-ray image data being monochrome into multicolor. Thus, it is favorable for enhancing the accuracy of the following analysis, but the present disclosure is not limited to the aforementioned description and the disclosure content of the drawings.

In Step 240, the normalized target hand bone X-ray image data is analyzed by the feature extracting module so as to obtain at least one image feature value. In detail, the method 200 for assessing the bone age and predicting the height of the present disclosure can automatically analyze the image information of the normalized target hand bone X-ray image data and obtain the corresponding image feature value by the feature extracting module, so that the assessing and predicting efficiency of the method 200 for assessing the bone age and predicting the height of the present disclosure can be enhanced.

In Step 250, the image feature value is analyzed by the bone age assessment and height prediction model so as to assess the development of the hand bone and the bone age of the subject and predict the adult height of the subject.

Please refer to FIG. 3, which is a block diagram of a bone age assessment and height prediction system 300 according to further another embodiment of the present disclosure. The bone age assessment and height prediction system 300 includes an image capturing unit 400 and a non-transitory machine readable medium 500.

The image capturing unit 400 is for obtaining a target hand bone X-ray image data of a subject, wherein the target hand bone X-ray image data includes a biological age data and a gender data. In detail, the image capturing unit 400 can be an X-ray examining device and uses a low-dose X-ray to illuminate the subject's hand so as to obtain a target hand bone X-ray image data with a proper resolution. More preferably, the target hand bone X-ray image data can be a target hand bone X-ray image data of a non-dominant hand so as to prevent the accuracy of the assessing results of the bone age assessment and height prediction system 300 of the present disclosure from affecting by the bone morphological variation caused by the using frequency and the using habits of the dominant hand. More preferably, a format of the target hand bone X-ray image data can be a DICOM format so as to store the biological age data, the gender data and other basic information of the target hand bone X-ray image data in the header files of the target hand bone X-ray image data and then facilitate the following analysis.

The non-transitory machine readable medium 500 is signally connected to the image capturing unit 400, wherein the non-transitory machine readable medium 500 is for storing a program (not shown), when the program executed by a processor (not shown) is for assessing a development of a hand bone and a bone age of a subject and predicting an adult height of the subject, and the aforementioned program includes a reference database obtaining module 510, a first image data editing module 520, a feature extracting module 530, a training module 540, a second image data editing module 550, a target feature extracting module 560 and a comparing module 570.

The reference database obtaining module 510 is for obtaining a reference database, and the reference database includes a bone age-height plotted chart database and a plurality of reference hand bone X-ray image data, wherein each of the reference hand bone X-ray image data includes a biological age data and a gender data. More preferably, each of the reference hand bone X-ray image data can be a reference hand bone X-ray image data of a non-dominant hand, and the bone age-height plotted chart database can include a male bone age-height plotted chart sub-database and a female bone age-height plotted chart sub-database so as to analyze the subjects of different genders.

More preferably, a format of the reference hand bone X-ray image data can be a DICOM format so as to store the biological age data, the gender data and other basic information of each of the reference hand bone X-ray image data in the header files of each of the reference hand bone X-ray image data and then facilitate the following analysis.

The first image data editing module 520 is for adjusting an image size and a monochrome contrast of each of the reference hand bone X-ray image data so as to obtain a plurality of normalized hand bone X-ray image data. In detail, the first image data editing module 520 can respectively adjust the image size of different reference hand bone X-ray image data into 256 pixels×256 pixels and adjust the monochrome contrast thereof, so that the color difference of black and white of different reference hand bone X-ray image data can be reduced and the image clarity can be enhanced so as to facilitate the following analysis.

Furthermore, each of the reference hand bone X-ray image data can be further processed by a color gamut expansion process by the first image data editing module 520. In detail, the first image data editing module 520 can calculate the gray level of each of the reference hand bone X-ray image data and automatically fill colors into pixel rows and pixel columns of image of each of the reference hand bone X-ray image data according to the aforementioned calculating results so as to transform each of the reference hand bone X-ray image data being monochrome into multicolor. Thus, it is favorable for enhancing the accuracy of the following analysis, but the present disclosure is not limited to the aforementioned description and the disclosure content of the drawings.

The feature extracting module 530 is for analyzing the normalized hand bone X-ray image data so as to obtain a plurality of reference image feature values, and each of the normalized hand bone X-ray image data corresponds to one of the reference image feature values. In detail, the bone age assessment and height prediction system 300 of the present disclosure can automatically analyze the image information of the normalized hand bone X-ray image data and obtain the corresponding image feature value by the feature extracting module 530.

The training module 540 is for training the reference image feature values to achieve a convergence of the reference image feature values by a convolutional neural network classifier so as to obtain a bone age assessment and height prediction model. More preferably, the convolutional neural network classifier can be Inception-ResNet-v2 convolutional neural network classifier so as to effectively expand the training depth of the convolutional neural network and then enhance the classification and the recognition of images of the training module 540.

The second image data editing module 550 is for adjusting an image size and a monochrome contrast of the target hand bone X-ray image data so as to obtain a normalized target hand bone X-ray image data. In detail, the second image data editing module 550 can adjust the image size of the target hand bone X-ray image data into 256 pixels×256 pixels and adjust the monochrome contrast thereof so as to enhance the image clarity, and then the aforementioned normalized target hand bone X-ray image data can be obtained. More preferably, the target hand bone X-ray image data can be further processed by a color gamut expansion process by the second image data editing module 550, wherein the gray level of the target hand bone X-ray image data can be calculated and then colors will be automatically filled in pixel rows and pixel columns of image of the target hand bone X-ray image data according to the aforementioned calculating results so as to transform the target hand bone X-ray image data being monochrome into multicolor, but the present disclosure is not limited to the aforementioned description and the disclosure content of the drawings.

The target feature extracting module 560 is for analyzing the normalized target hand bone X-ray image data so as to obtain at least one target image feature value. In detail, the target feature extracting module 560 of the present disclosure can automatically analyze the image information of the normalized target hand bone X-ray image data and obtain the corresponding image feature value. In particular, the target feature extracting module 560 can automatically divide a palm area and a background area of the target hand bone X-ray image data, wherein the palm area is served as a positive sample, and the areas expect for the palm area are served as negative samples. Next, the aforementioned positive sample and negative samples are analyzed by the target feature extracting module 560 so as to obtain the target image feature value of the normalized target hand bone X-ray image data and then process the following analysis.

The comparing module 570 is for analyzing the at least one target image feature value by the bone age assessment and height prediction model so as to obtain a target image feature weight data, wherein the target image feature weight data is compared to the bone age-height plotted chart database of the reference database so as to output an assessing result of the development of the hand bone of the subject, an assessing result of the bone age of the subject and a predicting result of an adult height of the subject.

Furthermore, because the physiological maturity of males and females are not the same, the developmental morphology of bones and their corresponding physiological ages are also different, so that the comparing module 570 can respectively compared the normalized target hand bone X-ray image data of different genders with the male bone age-height plotted chart sub-database or the female bone age-height plotted chart sub-database so as to assess and predict the development of the hand bone, the bone age and the adult height of the subject.

Furthermore, although the figures are not shown, the bone age assessment and height prediction system 300 of the present disclosure can further include a warning module (not shown). After the normalized target hand bone X-ray image data is compared with the bone age-height plotted chart database, if the assessing result of the bone age of the subject is ahead of or behind the physiological age thereof, the warning module can issue a proactive warning message at the first time so as to facilitate the implementation of subsequent treatment or other related response measures.

The present disclosure will be further exemplified by the following specific examples according to the aforementioned description.

### EXAMPLES

### I. Reference Database

The reference database used in the present disclosure is the retrospective pediatric bone age X-ray image data collected by China Medical University Hospital. This clinical trial program is approved by China Medical University & Hospital Research Ethics Committee, which is numbered as CMUH 107-REC2-097. The reference database includes reference hand bone X-ray image data of 2758 male subjects, 4462 female subjects, total of 7220 subjects, and the subjects are aged from 2 to 16 years old. The format of all the aforementioned reference hand bone X-ray image data is a DICOM format so as to store the biological age data, the gender data, the medical record number, the testing number and other information of each of the subjects in the header files of the image data and then facilitate the following analysis.

The aforementioned reference database also includes a bone age-height plotted chart database. In detail, the aforementioned reference hand bone X-ray image data is a reference hand bone X-ray image data of a non-dominant hand of the subject so as to prevent the credibility of the reference database from affecting by the bone morphological variation caused by the using frequency and the using habits of the dominant hand, and the bone age-height plotted chart database includes reference data, such as a bone growth atlas and a growth curve atlas. Furthermore, the bone age-height plotted chart database can include a male bone age-height plotted chart sub-database and a female bone age-height plotted chart sub-database so as to analyze the subjects of different genders.

### II. The Bone Age Assessment and Height Prediction Model of the Present Disclosure

Please refer to FIG. 4, which is a partial establishing flow chart of the bone age assessment and height prediction model (not shown) of the present disclosure. In the example of FIG. 4, a reference hand bone X-ray image data 611a, a reference hand bone X-ray image data 611b and a reference hand bone X-ray image data 611c, for example, are used to illustrate the operating method and the analyzing method of the bone age assessment and height prediction model of the present disclosure.

First, after obtaining the aforementioned reference database, the reference hand bone X-ray image data 611a, the reference hand bone X-ray image data 611b and the reference hand bone X-ray image data 611c are respectively processed by an image preprocessing step 620 so as to normalize the size and the color thereof and then obtain a normalized hand bone X-ray image data 621a, a normalized hand bone X-ray image data 621b and a normalized hand bone X-ray image data 621c. In detail, the image preprocessing step 620 adjusts the image size of the reference hand bone X-ray image data 611a, the reference hand bone X-ray image data 611b and the reference hand bone X-ray image data into 256 pixels×256 pixels and adjusts the monochrome contrast thereof by an image data editing module (not shown) so as to enhance the image clarity and reduce the color difference of black and white of different reference hand bone X-ray image data.

More preferably, each of the reference hand bone X-ray image data can be further processed by a color gamut expansion process by the image data editing module according to actual needs. The image data editing module calculates the gray level of each of the reference hand bone X-ray image data and automatically fills colors into pixel rows and pixel columns of image of the reference hand bone X-ray image data 611a, the reference hand bone X-ray image data 611b and the reference hand bone X-ray image data 611c, respectively, according to the aforementioned calculating results so as to transform each of the reference hand bone X-ray image data being monochrome into multicolor. Thus, the accuracy of the following analysis can be enhanced.

Furthermore, because the basic information, such as the biological age data and the gender data of each of the subjects are stored in the header files of the normalized hand bone X-ray image data 621a, the normalized hand bone X-ray image data 621b and the normalized hand bone X-ray image data 621c being a DICOM format, the bone age assessment and height prediction model of the present disclosure can directly extract the biological age data and the gender data of the normalized hand bone X-ray image data 621a, the normalized hand bone X-ray image data 621b and the normalized hand bone X-ray image data 621c, so that it is not needed to manually perform additional labeling operations, and is favorable for omitting additional analysis procedures and improving the analysis efficiency.

The normalized hand bone X-ray image data 621a, the normalized hand bone X-ray image data 621b and the normalized hand bone X-ray image data 621c processed by the aforementioned image preprocessing step 620 will be further processed by a feature extracting step 630, respectively, so as to be analyzed by a feature extracting module (not shown) and then obtain a plurality of image feature values, and each of the normalized hand bone X-ray image data 621a, the normalized hand bone X-ray image data 621b and the normalized hand bone X-ray image data 621c corresponds to one of the image feature values. In detail, the feature extracting module can respectively divide a palm area and a background area of the normalized hand bone X-ray image data 621a, the normalized hand bone X-ray image data 621b and the normalized hand bone X-ray image data 621c, wherein the palm area is served as a positive sample, and the areas expect for the palm area are served as negative samples. Next, the aforementioned positive sample and negative samples are analyzed by the feature extracting module so as to obtain the image feature values, respectively.

Furthermore, please refer to FIG. 4 and FIG. 5 simultaneously, and FIG. 5 is a block diagram of a convolutional neural network classifier 641 of the bone age assessment and height prediction model of the present disclosure. In the example of FIG. 5, the convolutional neural network classifier 641 is Inception-ResNet-v2 convolutional neural network classifier and includes a plurality of convolution layers (Convolution), a plurality of maximum pooling layers (MaxPool), a plurality of average pooling layers (AvgPool) and a plurality of contact layers (Concat) so as to train and analyze the image feature values.

In the process for training the image feature values first, the image feature values of the normalized hand bone X-ray image data 621a, the normalized hand bone X-ray image data 621b and the normalized hand bone X-ray image data 621c will be respectively processed by two convolution layers and one maximum pooling layer so as to maximum output the extracted image feature values. Then, the aforementioned training of two convolution layers and one maximum pooling layer are repeated and output, and then the extracted image feature values are trained by a plurality of convolution layers by a parallel towers method (parallel towers) so as to finish the primary training (Inception) of the image feature values.

After the aforementioned primary training is finished, the image feature values of the normalized hand bone X-ray image data 621a, the normalized hand bone X-ray image data 621b and the normalized hand bone X-ray image data 621c will be respectively trained by 10 times (10×), 20 times (20×) and 10 times (10×), 20 times (20×) and 10 times (10×) of different depths, different classes and different appearances of residual module training so as to train the image feature values to achieve a convergence thereof. In detail, by the training of the residual module, it is favorable for preventing the gradients of the aforementioned image feature values from disappearing or other degradations after training by the convolutional neural network classifier 641, and the training efficiency of the convolutional neural network classifier 641 can be effectively enhanced.

After the deep and repeated training of the residual module is finished, the convergent image feature values will be finally trained and processed by one convolution layer, one average pooling layer, one global average pooling layer (Global Average Pooling 2D, GloAvePool2D) and one rectified linear unit layer (Rectified Linear Unit, ReLU) sequentially so as to assess the development of the hand bone and the bone age of the subject and predict the adult height of the subject. Wherein, the average pooling layer can calculate the image feature values trained by the residual module first so as to obtain an average value of each of the image feature values, and the global average pooling layer can process a regularization method (Regularization) to the overall network structure of the convolutional neural network classifier 641 so as to prevent the overfitting condition (Overfitting) of the convolutional neural network classifier 641 under the training mode which is pursuing low error, resulting in high error values of the assessing result, which makes the results of the bone age assessment and height prediction model less reliable than expected. Finally, the rectified linear unit layer will further activate the trained image feature value and then output a target image feature weight data 650 so as to process the following comparison and analysis. The aforementioned rectified linear unit layer can prevent the target image feature weight data 650 output from the bone age assessment and height prediction model from approaching zero or approaching infinity, so that it is favorable for processing the following analyzing steps, and then the assessing accuracy of the bone age assessment and height prediction model of the present disclosure can be enhanced.

### III. The Bone Age Assessment and Height Prediction Model of the Present Disclosure for Assessing a Development of a Hand Bone and a Bone Age of a Subject and Predicting an Adult Height of the Subject

In the present example, the established bone age assessment and height prediction model will be further used to assess a development of a hand bone and a bone age of a subject and predict an adult height of the subject. The steps thereof are shown as follow: the bone age assessment and height prediction model established above is provided. A target hand bone X-ray image data of a subject is provided, wherein the target hand bone X-ray image data includes a biological age data and a gender data. The target hand bone X-ray image data is preprocessed, wherein an image size and a monochrome contrast of the target hand bone X-ray image data are adjusted by the image data editing module so as to obtain a normalized target hand bone X-ray image data. The normalized target hand bone X-ray image data is analyzed by the feature extracting module so as to obtain at least one image feature value. The image feature value is analyzed by the bone age assessment and height prediction model so as to assess the development of the hand bone and the bone age of the subject and predicting the adult height of the subject.

Furthermore, the bone age assessment and height prediction model established above will be applied in the bone age assessment and height prediction system of the present disclosure so as to integrate the assessing result of the development of the hand bone of the subject, the assessing result of the bone age of the subject, and the predicting result of the adult height of the subject into the reference database, so that the bone age assessment and height prediction model can be optimized. Furthermore, the details of the structure of the bone age assessment and height prediction system of the present disclosure are described in FIG. 3 and the aforementioned description and are not described again herein.

Please refer to FIG. 6, which shows an applying result 700 of the bone age assessment and height prediction system (not shown) of the present disclosure. The bone age assessment and height prediction model of the bone age assessment and height prediction system will further output an assessing result of the development of the hand bone of the subject and an assessing result of the bone age of the subject after finishing the analysis, and the results will be shown on a display module (not shown). As shown in FIG. 6, the applying result 700 of the bone age assessment and height prediction system can include a result column 701, a result column 702, a result column 703 and a result column 704. The result column 701 can show the basic information of the subject, including the biological age data, the gender data, the medical record number, the testing number and other personal information, the result column 702 shows the target hand bone X-ray image data of the subject before preprocessing, the result column 703 shows a bone age of the subject assessed by the bone age assessment and height prediction model, and result column 704 shows the atlas of bone age before and after 12 months the aforementioned bone age of the subject assessed by the bone age assessment and height prediction model so as to facilitate the comparison and analysis of the analyzer.

Furthermore, although the figures are not shown, the bone age assessment and height prediction system of the present disclosure can further compare the assessing result of the development of the hand bone age of the subject and the assessing result of the bone age of the subject with the male bone age-height plotted chart sub-database or the female bone age-height plotted chart sub-database of the bone age-height plotted chart database so as to predict an adult height of the subjects of different genders, and then a predicting result of an adult height of the subject will be output simultaneously and displayed in the aforementioned display module, and the present disclosure is not limited to the aforementioned description and the disclosure content of the drawings.

Furthermore, although the figures are not shown, the bone age assessment and height prediction system of the present disclosure can further include a warning module (not shown). After the bone age assessment and height prediction model outputs the assessing result of the bone age of the subject, if the assessing results of the bone age of the subject is ahead of or behind the physiological age thereof, the warning module can issue a proactive warning message at the first time and the warning message will be shown by red words in the result column 703 so as to facilitate the implementation of subsequent treatment or other related response measures.

Therefore, the bone age assessment and height prediction model, the bone age assessment and height prediction system and the method for assessing a bone age and predicting a height of the present disclosure can automatically extract the image feature value of the target hand bone X-ray image data of the subject and process a deep neural network training by the bone age assessment and height prediction model. Not only it is favorable for shortening the time period for assessing the bone age and predicting the height, but also the result errors generated by the different feature selection and comparison methods of different analysts in the conventional bone age assessment methods can be avoided. Furthermore, the bone age assessment and height prediction model including the convolutional neural network classifier can efficiently enhance the accuracy and the sensitivity of the assessment of bone age and the prediction of the height. Thus, the bone age assessment and height prediction model, the bone age assessment and height prediction system and the method for assessing a bone age and predicting a height have a better efficiency for assessing the bone age and predicting the height, and a treatment or related application measures can be implemented according to the assessing result of the bone age of the subject so as to reduce the incidence of children's diseases caused by stunting or precocious puberty.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

1. A bone age assessment and height prediction model established by following steps:
obtaining a reference database, wherein the reference database comprises a bone age-height plotted chart database and a plurality of reference hand bone X-ray image data, and each of the reference hand bone X-ray image data comprises a biological age data and a gender data;
performing an image preprocessing step, wherein an image size and a monochrome contrast of each of the reference hand bone X-ray image data are adjusted by an image data editing module so as to obtain a plurality of normalized hand bone X-ray image data;
performing a feature extracting step, wherein the normalized hand bone X-ray image data are analyzed by a feature extracting module so as to obtain a plurality of image feature values, and each of the normalized hand bone X-ray image data corresponds to one of the image feature values; and
performing a training step, wherein the image feature values are trained to achieve a convergence of the image feature values by a convolutional neural network classifier so as to obtain the bone age assessment and height prediction model, wherein the bone age assessment and height prediction model is for assessing a development of a hand bone and a bone age of a subject and predicting an adult height of the subject.

2. The bone age assessment and height prediction model of claim 1, wherein the convolutional neural network classifier is Inception-ResNet-v2 convolutional neural network classifier.

3. The bone age assessment and height prediction model of claim 1, wherein a format of each of the reference hand bone X-ray image data is a DICOM (Digital Imaging and Communications in Medicine) format.

4. The bone age assessment and height prediction model of claim 1, wherein each of the reference hand bone X-ray image data is further processed by a color gamut expansion process in the image preprocessing step.

5. The bone age assessment and height prediction model of claim 1, wherein the bone age-height plotted chart database comprises a male bone age-height plotted chart sub-database and a female bone age-height plotted chart sub-database.

6. The bone age assessment and height prediction model of claim 1, wherein each of the reference hand bone X-ray image data is a reference hand bone X-ray image data of a non-dominant hand.

7. A method for assessing a bone age and predicting a height, comprising:
providing the bone age assessment and height prediction model of claim 1;
providing a target hand bone X-ray image data of the subject, wherein the target hand bone X-ray image data comprises a biological age data and a gender data;
preprocessing the target hand bone X-ray image data, wherein an image size and a monochrome contrast of the target hand bone X-ray image data are adjusted by the image data editing module so as to obtain a normalized target hand bone X-ray image data;
analyzing the normalized target hand bone X-ray image data by the feature extracting module so as to obtain at least one image feature value; and
analyzing the at least one image feature value by the bone age assessment and height prediction model so as to assess the development of the hand bone and the bone age of the subject and predicting the adult height of the subject.

8. The method for assessing the bone age and predicting the height of claim 7, wherein a format of the target hand bone X-ray image data is DICOM format.

9. The method for assessing the bone age and predicting the height of claim 7, wherein the target hand bone X-ray image data is further processed by a color gamut expansion process by the image data editing module.

10. The method for assessing the bone age and predicting the height of claim 7, wherein the target hand bone X-ray image data is a target hand bone X-ray image data of a non-dominant hand.

11. A bone age assessment and height prediction system, comprising:
an image capturing unit for obtaining a target hand bone X-ray image data of a subject, wherein the target hand bone X-ray image data comprises a biological age data and a gender data; and
a non-transitory machine readable medium signally connected to the image capturing unit, wherein the non-transitory machine readable medium is for storing a program, when the program executed by a processor is for assessing a development of a hand bone and a bone age of a subject and predicting an adult height of the subject, and the program comprises:
a reference database obtaining module for obtaining a reference database, wherein the reference database comprises a bone age-height plotted chart database and a plurality of reference hand bone X-ray image data, and each of the reference hand bone X-ray image data comprises a biological age data and a gender data;
a first image data editing module for adjusting an image size and a monochrome contrast of each of the reference hand bone X-ray image data so as to obtain a plurality of normalized hand bone X-ray image data;
a feature extracting module for analyzing the normalized hand bone X-ray image data so as to obtain a plurality of reference image feature values, and each of the normalized hand bone X-ray image data corresponds to one of the reference image feature values;
a training module for training the reference image feature values to achieve a convergence of the reference image feature values by a convolutional neural network classifier so as to obtain a bone age assessment and height prediction model;
a second image data editing module for adjusting an image size and a monochrome contrast of the target hand bone X-ray image data so as to obtain a normalized target hand bone X-ray image data;
a target feature extracting module for analyzing the normalized target hand bone X-ray image data so as to obtain at least one target image feature value; and
a comparing module for analyzing the at least one target image feature value by the bone age assessment and height prediction model so as to obtain a target image feature weight data, wherein the target image feature weight data is compared to the bone age-height plotted chart database of the reference database so as to output an assessing result of the development of the hand bone of the subject, an assessing result of the bone age of the subject and a predicting result of an adult height of the subject.

12. The bone age assessment and height prediction system of claim 11, wherein the convolutional neural network classifier is Inception-ResNet-v2 convolutional neural network classifier.

13. The bone age assessment and height prediction system of claim 11, wherein a format of the target hand bone X-ray image data is a DICOM format, and a format of each of the reference hand bone X-ray image data is a DICOM format.

14. The bone age assessment and height prediction system of claim 11, wherein each of the reference hand bone X-ray image data is further processed by a color gamut expansion process by the first image data editing module, and the target hand bone X-ray image data is further processed by a color gamut expansion process by the second image data editing module.

15. The bone age assessment and height prediction system of claim 11, wherein the bone age-height plotted chart database comprises a male bone age-height plotted chart sub-database and a female bone age-height plotted chart sub-database.

16. The bone age assessment and height prediction system of claim 11, wherein each of the reference hand bone X-ray image data is a reference hand bone X-ray image data of a non-dominant hand, and the target hand bone X-ray image data is a target hand bone X-ray image data of a non-dominant hand.

17. The bone age assessment and height prediction system of claim 11, further comprising:
a warning module for providing an active warning message after the at least one target image feature value of the normalized target hand bone X-ray image data being analyzed by the bone age assessment and height prediction model.
